(19) United States
(12) Patent Application Publication
(10) Pub. No.: US 2026/0320476 A1
(43) Pub. Date: Oct. 15, 2026

(54) SYSTEM AND METHOD FOR DYNAMIC FORMULATION ACTIVE NATURAL OIL-BASED BIOPESTICIDES

(71) Applicants: Nestor Leiderman, Beer Sheva (IL); Daniel Noah Plewinski, Meitar (IL); Daniel Halevi, Kfar Saba (IL)

(72) Inventors: Nestor Leiderman, Beer Sheva (IL); Daniel Noah Plewinski, Meitar (IL); Daniel Halevi, Kfar Saba (IL)

(21) Appl. No.: 19/175,021

(22) Filed: Apr. 10, 2025

(51) Int. Cl. G06N 20/00; A01N 65/26; A01P 3/00

(52) U.S. Cl. CPC G06N 20/00; A01N 65/26; A01P 3/00

(57) ABSTRACT

The subject matter discloses system and method for dynamic formulation of biopesticides based on active natural oils. The system personalizes each formulation according to crop type, target pests or fungi, infestation severity, and real-time environmental parameters such as temperature and humidity. Using a combination of effectiveness matrices and non-linear sigmoid-based adjustments, the system calculates optimized ratios for each oil. These ratios are then normalized to comply with regulatory limits, including FIFRA 25(b) exemptions. The output includes a structured data file or executable code for automated machines that produce the final product and generate compliant labels. The system enables precision agriculture with tailored biopesticide mixtures that maintain regulatory compliance, reduce overuse, and increase efficacy in complex multi-infestation scenarios.

### FIELD OF THE INVENTION

The present disclosure relates to agricultural technology in general, and to formulations, in particular.

### BACKGROUND OF THE INVENTION

Emulsifiable Concentrates (EC) are oil-based liquids that mix with water to form a sprayable emulsion and include a fixed amount of oil. Examples of such formulations include:
Neemazal-T/S (Trifolio-M GmbH, Germany)
   Type: Emulsifiable Concentrate (EC)
   Active Ingredient: Azadirachtin (Neem oil extract)
   Mode of Action: Insect growth regulator (IGR), anti-feedant, and repellent
Voliam Targo (Syngenta)
   Type: Oil Dispersion (OD)
   Active Ingredients: Chlorantraniliprole+Abamectin
   Mode of Action: Targets caterpillars and mites in vegetables, fruits, and tea plantations
Polo 500 EC (Adama)
   Type: Emulsifiable Concentrate (EC)
   Active Ingredient: Diafenthiuron (500 g/L)
   Mode of Action: Broad-spectrum insecticide effective against resistant whiteflies and thrips
   These examples illustrate conventional oil-based emulsifiable concentrates

### SUMMARY OF THE INVENTION

The term “computing device” refers to a device that includes a processing unit. Examples include a personal computer, a laptop, a server, a wearable device, a tablet, a cellular device, and an IoT (Internet of Things) device.

The term “personalize” refers to tailoring the formulation of a combination of active natural oil-based biopesticides to specific elements affecting biopesticide efficiency. These elements include crop type, target pests or fungi, infestation severity, environmental conditions at the time of application, and the percentage of dilution of the natural oils.

The term data structure refers herein to a file that includes information in a data interchange format. The file is designed for transmitting data between systems, typically over the internet. An example for such a file is the JSON file.

Embodiments of the invention provide a system and method for the dynamic formulation of active natural oil-based biopesticides. The system incorporates environmental and agricultural data to optimize oil concentrations, targeting specific pests and diseases, including multiple simultaneous infestations, while remaining compliant with regulations such as FIFRA 25(b). The formulation utilizes active natural oils to address single and multiple pest and fungal infestations across various crops.

The system generates personalized formulations based on real-time agricultural and environmental data. It accommodates multiple infestations, adjusting oil concentrations to balance efficacy across pest types, and generates compliant labels for biopesticides.

### Example of a Dynamic Formulation Calculation

Inputs:
   Crop: Tomato
   Pests: Aphids (Moderate), Fungi (Severe)
   Temperature: 30 °C.
   Humidity: 70%
   Calculation:
   Neem Oil:
      Base: 30%
      Aphid Preference: 1.5×30%=45%
      Severity Adjustment (Moderate): 45%×1.0=45%
      Temperature Adjustment (Sigmoid Function Applied): +0.02×Sigmoid(30°)
      Humidity Adjustment (Sigmoid Function Applied): +0.01×Sigmoid(70%)
      Final Ratio: 54.24%
   Thyme Oil:
      Base: 15%
      Fungi Preference: 2.0×15%=30%
      Severity Adjustment (Severe): 30%×1.2=36%
      Temperature Adjustment (Sigmoid Function Applied): +0.025×Sigmoid(30°)
      Humidity Adjustment (Sigmoid Function Applied): +0.02×Sigmoi(70%)
      Final Ratio: 45.76%
      Other Oils:
      Calculated similarly, adjusted for lower preference scores.
   Output:
      Neem Oil: 54.24%
      Thyme Oil: 45.76%

One technical problem disclosed by the present disclosure is how to take into account the specific pests and diseases, the specific crop and the environmental condition in the formulation of Biopesticides. Current pesticide application methods in agriculture lack personalization, leading to overuse of chemicals, decrease efficiency and may cause potential environmental harm. Generic formulations frequently fail to address specific agricultural needs, particularly in cases involving unique or multiple infestations under varying environmental conditions. The methods known in the art provide uniform formulation and are thus inflexible and inefficient since they do not handle crop-specific or regional pest challenges. Such methods do not address infestation severity or the presence of multiple pests or fungi. Known in the art formulations illustrate conventional oil-based emulsifiable concentrates, which, unlike the present disclosure, lack dynamic adjustment capabilities based on environmental conditions, crop type, and pest severity.

One technical solution is the use of a dynamic computational model that adjusts the formulation based on real-time environmental and agricultural data. The system processes inputs such as crop type, pest species, infestation severity, temperature, and humidity, dynamically recalculating oil concentrations to optimize efficacy. Additionally, it ensures compliance with FIFRA 25(b) by automatically generating legally compliant labels for the final formulation. Unlike existing formulation approaches, which primarily consider static parameters for specific crops or pests, the present disclosure enables real-time dynamic adjustment of oil concentrations based on multiple environmental factors crop type, and pest severity.

One other technical problem is how to comply with regulatory constraints, such as FIFRA 25(b), which demand strict compliance for pesticide labelling and composition.

One other technical solution is to use natural oils to ensure regulatory compliance by dynamically adjusting the formulation to meet FIFRA 25(b) ingredient and concentration restrictions. Additionally, the system generates code or data structure for generating compliant labels based on the selected oils and their ratios, ensuring that all regulatory requirements are met.

In some aspects of the present invention relates to a non-transitory computer-readable medium comprising instructions which when executed by at least one processor causes the processor to perform the method of the present invention.

One exemplary embodiment of the disclosed subject matter is a method for dynamic formulation of active natural oil-based, the method comprises:
receiving, from a user interface, one or more types of pests or fungus, ratio of one or more non-active ingredient and one or more parameters associated with environmental conditions or with the pest or fungus; for each of the one or more types of pests or fungus, retrieving from a data repository, one or more effective oil-types; retrieving from the data repository effective base ratio associated with each of the pest or fungus; adjusting the effective base ratio in accordance with the one or more environmental parameters to thereby providing a plurality of adjusted effective base ratio; normalizing the plurality of adjusted effective base ratio; generating a data structure or software code; the data structure comprises the type of pest or fungus, the oil types associated with the normalized value and the one or more non-active ingredient in association with its ratio; the software code comprises instructions for generating labels for the active natural oil-based or instructions for generating the active natural oil-based; and transmitting the data structure to a machine for generating labels for the product or for the machine for generating the active natural oil-based or transmitting the software code to a machine for generating labels for the product or the machine for generating the active natural oil-based. According to some embodiments the one or more parameters comprises one member selected from a group consisting of: infestation severity of each of the pet or fungus, humidity level and temperature level.

One other exemplary embodiment of the disclosed subject matter is a method for dynamic formulation of active natural oil-based, the method comprises: receiving, from a user interface, one or more types of pests or fungus, one or more parameters associated with environmental conditions or with the pest or fungus and ratio of one or more non-active ingredient; for each of the one or more types of pests or fungus, retrieving from a data repository, one or more effective oil-types; retrieving from the data repository effective base ratio associated with each of the pest or fungus and with each of the one or more effective oil-type; accumulating effective base ratio associated with same oil type from the plurality of oil types to, thereby, generating a plurality of accumulated effective base ratios; for each of the plurality of accumulative base ration adjusting the accumulative base ratio, in accordance with the one or more parameters, normalizing each of the adjusted accumulative base ratio; generating a data structure or software code; the data structure or the software code comprises the type of pest or fungus, the oil types associated with the normalized value and the one or more non-active ingredient in association with its ratio; and
transmitting the data structure or the software code to a machine for generating labels for the product or for the machine for generating the active natural oil-based biopesticides.

The method of claim 3, wherein the one or more parameters comprises one member selected from a group consisting of: infestation severity of each of the pet or fungus, humidity level and temperature level.

According to some embodiments the adjusting being by a non-linear optimization function. According to some embodiments the non-linear optimization function being sigmoid based module. One other exemplary embodiment of the disclosed subject matter is a computer readable storage medium having encoded thereon computer executable instructions to cause one or more processing units to receive, from a user interface, one or more types of pests or fungus, ratio of one or more non-active ingredient and one or more parameters associated with environmental conditions or with the pest or fungus; for each of the one or more types of pests or fungus, retrieve from a data repository, one or more effective oil-types; retrieve from the data repository effective base ratio associated with each of the pest or fungus; adjust the effective base ratio in accordance with the one or more environmental parameters to thereby providing a plurality of adjusted effective base ratio; normalizing the plurality of adjusted effective base ratio; generate a data structure or software code; the data structure comprises the type of pest or fungus, the oil types associated with the normalized value and the one or more non-active ingredient in association with its ratio; the software code comprises instructions for generating labels for the active natural oil-based or instructions for generating the active natural oil-based; and transmit the data structure to a machine for generating labels for the product or for the machine for generating the active natural oil-based or transmitting the software code to a machine for generating labels for the product or the machine for generating the active natural oil-based. One other exemplary embodiment of the disclosed subject matter is a personalized composition comprising a plurality of oils, the oils being effective for a certain pest or fungus wherein ratio of each of the oils in the mixture being defined by effectiveness of the oil in treating the pest or fungus and further being defined by one or more parameters associated with environmental conditions or with the pest or fungus. According to some embodiments the one or more parameters comprises one member selected from a group consisting of: infestation severity of each of the pet or fungus, humidity level and temperature level. One other exemplary embodiment of the disclosed subject matter is a method comprising applying to a crop the composition described herein.

Embodiments of the invention may be implemented as a computer process (method), a computing system, or as an article of manufacture, such as a computer program product or a non-transitory computer-readable media. The computer program product may be a computer storage media readable by a computer system and encoding a computer program of instructions for executing a computer process on the computer and network devices. The computer program product may also be a propagated signal on a carrier readable by a computing system and encoding a computer program of instructions for executing a computer process.

### THE BRIEF DESCRIPTION OF THE SEVERAL VIEWS OF THE DRAWINGS

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIG. 1 shows block diagram of an environment for dynamic formulation of active oil-based biopesticides, in accordance with some exemplary embodiments of the subject matter;

FIG. 2 shows an exemplary data structure of properties of oils, in accordance with some exemplary embodiments of the disclosed subject matter;

FIG. 3A shows an exemplary data structure of pests preferences, in accordance with some exemplary embodiments of the disclosed subject matter;

FIG. 3B shows an exemplary data structure of pest severity multiplier, in accordance with some exemplary embodiments of the disclosed subject matter; and

FIG. 4 shows a block diagram of a method for dynamic formulation of active natural oil-based biopesticides.

### DETAILED DESCRIPTION

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

If the performance of an operation is described herein as being “based on” one or more factors, it is to be understood that the performance of the operation may be based solely on such factor(s) or may be based on such factor(s) along with one or more additional factors. Thus, as used herein, the term “based on” should be understood to be equivalent to the term “based at least on.”

Numerous exemplary embodiments are now described. Any section/subsection headings provided herein are not intended to be limiting. Embodiments are described throughout this document, and any type of embodiment may be included under any section/subsection. Furthermore, embodiments disclosed in any section/subsection may be combined with any other embodiments described in the same section/subsection and/or a different section/subsection in any manner.

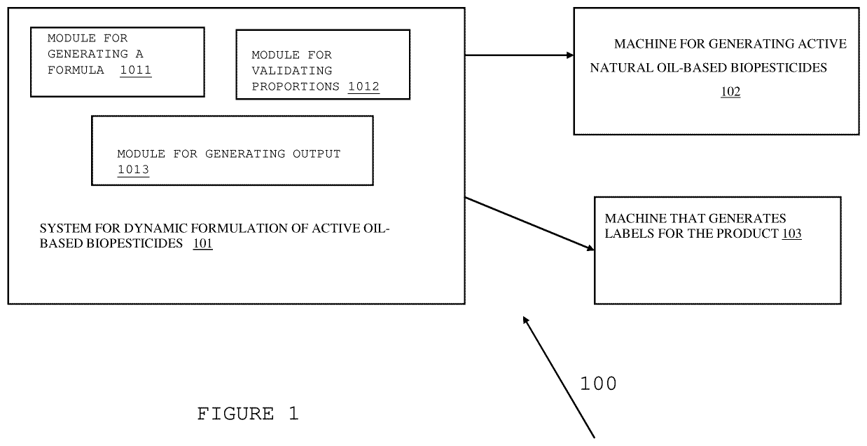

Referring now to FIG. 1 showing block diagram of an environment for dynamic formulation of active oil-based biopesticides, in accordance with some exemplary embodiments of the subject matter.

Environment 100 includes a system for generating dynamic formulation of active oil-based biopesticides 101, a machine for generating active natural oil-based biopesticides 102 and a machine for generating labels for product 103.

The system for generating dynamic formulation of active oil-based biopesticides 101 is configured for generating personalized formulas. The system for generating dynamic formulation of active oil-based biopesticides 101 includes a module for generating formula 1011, a module for validating proportions 1012 and a module for generating output 1013.

The module for generating formula 1011 is configured for dynamically defining the personalized formula according to input parameters.

The module for validating proportions 1012 is configured for normalizing the values of the formula. If the validation module (1012) detects that the total proportion of active oils does not meet the required balance (e.g., exceeding regulatory thresholds), it returns feedback to the formulation module (1011) for recalculation. This iterative process ensures that the final formulation adheres to regulatory requirements while maintaining optimal efficacy.

The module for generating output 1013 is configured for generating data structure and/or code to be transferred to the machine for generating active natural oil-based biopesticides 102 and to the machine for generating labels for product 103.

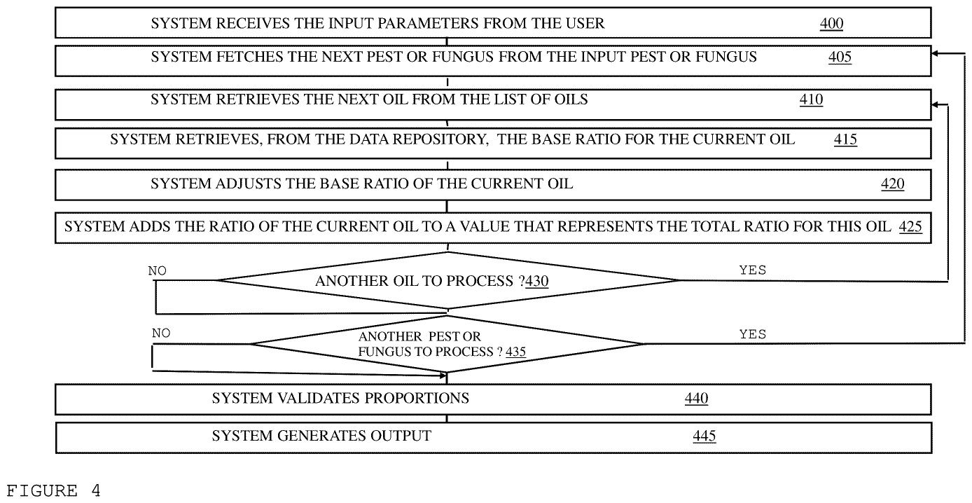

The operation of the modules is explained in greater detail in FIG. 4.

In some embodiments the system for generating dynamic formulation of active oil-based biopesticides 101 is communicatively coupled to the machine for generating active natural oil-based biopesticides 102 by one or more communications channels. The coupling is for transmitting the code for generating the formula of natural oil-based biopesticides.

In some embodiments the system for generating dynamic formulation of active oil-based biopesticides 101 is communicatively coupled to the machine for generating labels for product 103 by one or more communications channels. The coupling is for transmitting the data structure for generating the labels.

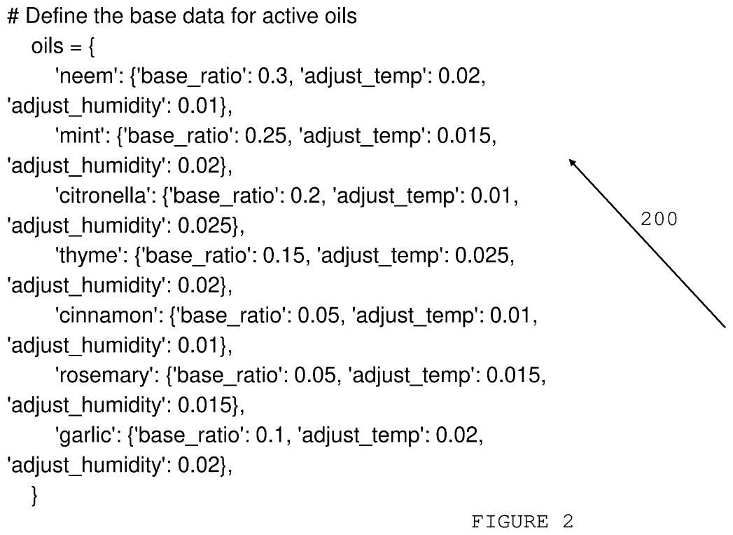

FIG. 2 shows an exemplary data structure of properties of oils, in accordance with some exemplary embodiments of the disclosed subject matter.

Structure 200 includes a list of oils. Per each oil the structure includes the basic ratio and parameters for adjusting to temperature and humidity.

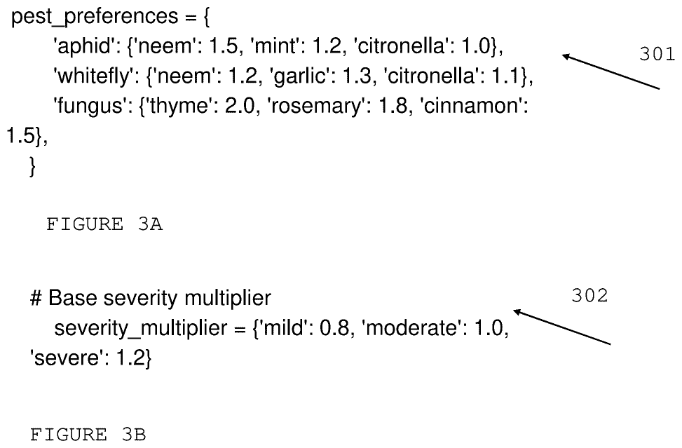

FIG. 3A shows an exemplary data structure of pests'preferences, in accordance with some exemplary embodiments of the disclosed subject matter. Structure 301 includes a list of pests. Each line defines a pest. Each pest includes a list of oils, wherein each oil in the list is associated with the preference related to this pest. The preference is a multiplier and is used for multiplying the base ratio of this oil with this preference.

FIG. 3B shows an exemplary data structure of pest severity multipliers, in accordance with some exemplary embodiments of the disclosed subject matter. Structure 302 includes a list of severity multipliers that are used for multiplying the base ratio of each oil that is included in the formula according to the severity level. In this example if the severity level is mild, the base ratio is multiplied by 0.8; if the severity level is moderate the base ratio is multiplied by 1.0 and if the severity level is mild the base ratio is multiplied by 1.2.

FIG. 4 shows a block diagram of a method for dynamic formulation of active natural oil-based biopesticides.

At block 400 the system receives the input parameters from the user. The input parameters include:

Type of crop to be protected by the formulation, for example, Tomatoes and roses.

Type of pest or fungus that attack the crop, For example Aphididae (Aphids) and Whiteflies.

Infestation severities of each pet or fungus. In some embodiments, severity is defined as mild, moderate and severe.

Temperature levels at the time of protecting the crop. Examples of such temperature levels are 10 Celsius to 20 Celsius and 20 Celsius to 40 Celsius.

Humidity levels at the time of protecting the crop. Examples of such humidity levels in percentage. are 60-70 and 50-60.

Non-Active ingredient: the list of the non-active ingredients each associated with a certain ratio. An example of such a list is water 10% and soy oil 20%.

At block 405 the system fetches the next pest or fungus from the input pest or fungus.

Blocks 410, 415, 420 and 425 are performed for each oil in the list of oils that are relevant to be used as biopesticides against the current handled pest or fungus.

At block 410 the system retrieves the next oil from the list of oils that are relevant to being used as biopesticides against the current handled pest or fungus. An example of a data structure that stores such information in the data repository is depicted in FIG. 3A. Examples of such oils are Neem oil, Peppermint oil and Garlic oil. In some embodiments the oils a nature oils for ensuring compliance with regulatory frameworks such as FIFRA 25(b).

At block 415 the system retrieves, from the data repository, the base ratio for the current oil. An example of a data structure that stores the base ratio per each oil is depicted in FIG. 2. Examples of such base ratios are:
Neem: 30%.
Peppermint: 25%.
Garlic: 10%.

At block 420 the system adjusts the base ratio of the current oil according to the effectiveness of this oil on the current handled pest or fungus and according to the severity of the damage. The data structure that stores effective value that is defined for this oil in relation to the current handled pest or fungus is retrieved from the data repository. An example of a data structure that stores effectiveness value that is defined for this oil in relation to the current handled pest or fungus is depicted in FIG. 3A. In some embodiments the ratio for the current oil is multiplied by its effectiveness score for the given pest.

Examples of such preference values is:
Aphids: Neem (1.5), Peppermint (1.2)
Fungus: Thyme (2.0), Rosemary (1.8)

The data structure that stores the effect of the input infestation severities on the ratio of this oil is retrieved from the data repository. In some embodiments the ratio for the current oil is multiplied by the effect of the input severity level of the input infestation severities on the ratio of this oil as depicted in FIG. 3B.

Examples of the effect of the severity level are:
Mild: Base×0.8.
Moderate: Base×1.0.
Severe: Base×1.2.

In some embodiments the system adjusts the ratio for the current oil to the temperature and or to the humidity level at the time of spraying the biopesticides.

In some embodiments, if the input temperature is below a predefined value, the system increases the ratio of this pet or fungus and if the input temperature is below a certain value the system decreases the ratio of this pet or fungus.

In some embodiments the system takes into account the humidity level that impacts on the effectiveness of antifungal and insecticidal properties. Oils with antifungal properties (e.g., Thyme, Rosemary) are boosted in high humidity. In such cases, if the input humidity exceeds a threshold, the system adjusts ratio of this oil accordingly.

An example of a data structure that stores percentage of increasing or decreasing the ratio value for this oil in relation to the humidity or temperature is depicted in FIG. 2.

An example of such calculation is:
Temperature Adjustment (Sigmoid Function Applied): +0.025×Sigmoid(30°)
Humidity Adjustment (Sigmoid Function Applied): +0.02×Sigmoi(70%)

The sigmoid function ensures a smooth, nonlinear adjustment, preventing excessive changes while maintaining responsiveness to environmental conditions.

At block 425, the system adds the ratio of the current oil to a value that represents the total ratio for this oil for all the input pest or fungus.

For a plurality of input pests or fungus, the system calculates a cumulative score for each oil by summing its contributions across all pests.

At block 430, the system checks if there is another oil to process for the current input of pest or fungus. If there is an oil the operation resumes to block 410 otherwise operation continues to block 435.

At block 435, the system checks if there is another pest or fungus to process if so, the operation resumes to block 405, otherwise the operation continues to block 440.

Blocks 440 illustrates the process of validating proportions. At block 440, the system normalizes the total ratio for all active oils to ensure that their sum equals the designated percentage for active ingredients. The normalization process follows the equation:

Normalized_ratio(oil)=(Raw_Ratio(oil)/ΣRaw_Ratios)×Total_Active_Percentage

Where:
Raw_Ratio(oil) is the adjusted ratio of the oil after environmental and pest-based calculations.
ΣRaw_Ratios is the sum of all raw ratios before normalization.
Total_Active_Percentage is the target percentage allocated to active oils (e.g., 80% if non-active ingredients make up 20%).

This method ensures that all active oils maintain their relative proportions while fitting within the required concentration limits and their relative distribution. It follows the equation: Normalized_Ratio(oil)=(Raw_Ratio(oil)/ΣRaw_Ratios)×Total_Active_Percentage Where: Raw_Ratio(oil) refers to the initial calculated proportion of a specific active oil before normalization. ΣRaw_Ratios represents the sum of all raw active oil ratios in the formulation. Total_Active_Percentage define the target percentage allocated to active oils (e.g., 80% if non-active ingredients make up 20%).

Block 445 illustrates the process of generating output. At block 445, the system generates a data structure for being used as an input for a machine that generates labels for the product and for the machine that generates the product. In one embodiment the labels are compliant with FIFRA (Federal Insecticide, Fungicide, and Rodenticide Act) 25(b). “”FIFRA 25(b) regulates pesticides in the U.S. Section 25(b) provides an exemption for minimum-risk pesticides that meet specific criteria. The generated data structure utilizes the total normalized values of each active oil and the input values of the non-active oils. In some embodiments the data structure is in a form of JSON (Java script Object notation) file. The data structure includes the normalized formula. In some embodiments the system generates software code for being used as an input for a machine that generates labels for the product and for the machine that generates the product. In some embodiments the software code is generating by an AI tool such as the chat GPT. The code includes instructions for generating the normalise formula or for generating the labels.

In some embodiments the system translates the ratio values into weight values such as grams for generating the code or the data structure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It should be noted that, in some alternative implementations, the functions noted in the block of a figure may occur out of the order noted in the figures. For example, two blocks shown in succession may, in fact, be executed substantially concurrently, or the blocks may sometimes be executed in the reverse order, depending upon the functionality involved.

What is claimed is:

1. A method for dynamic formulation of active natural oil-based, the method comprises:
receiving, from a user interface, one or more types of pests or fungus, ratio of one or more non-active ingredient and one or more parameters associated with environmental conditions or with said pest or fungus;
for each of said one or more types of pests or fungus, retrieving from a data repository, one or more effective oil-types
retrieving from said data repository effective base ratio associated with each of said pest or fungus;
adjusting said effective base ratio in accordance with said one or more environmental parameters to thereby providing a plurality of adjusted effective base ratio;
normalizing said plurality of adjusted effective base ratio;
generating a data structure or software code; said data structure comprises said type of pest or fungus, said oil types associated with said normalized value and said one or more non-active ingredient in association with its ratio; said software code comprises instructions for generating labels for said active natural oil-based or instructions for generating said active natural oil-based; and
transmitting said data structure to a machine for generating labels for the product or for said machine for generating said active natural oil-based or transmitting said software code to a machine for generating labels for the product or said machine for generating said active natural oil-based.

2. The method of claim 1 wherein said one or more parameters comprises one member selected from a group consisting of: infestation severity of each of said pet or fungus, humidity level and temperature level.

3. A method for dynamic formulation of active natural oil-based, the method comprises:
receiving, from a user interface, one or more types of pests or fungus, one or more parameters associated with environmental conditions or with said pest or fungus and ratio of one or more non-active ingredient;
for each of said one or more types of pests or fungus, retrieving from a data repository, one or more effective oil-types;
retrieving from said data repository effective base ratio associated with each of said pest or fungus and with each of said one or more effective oil-type;
accumulating effective base ratio associated with same oil type from said plurality of oil types to, thereby, generating a plurality of accumulated effective base ratios;
for each of said plurality of accumulative base ration adjusting said accumulative base ratio, in accordance with said one or more parameters,
normalizing each of said adjusted accumulative base ratio;
generating a data structure or software code; said data structure or said software code comprises said type of pest or fungus, said oil types associated with said normalized value and said one or more non-active ingredient in association with its ratio; and
transmitting said data structure or said software code to a machine for generating labels for the product or for the machine for generating said active natural oil-based biopesticides.

4. The method of claim 3, wherein said one or more parameters comprises one member selected from a group consisting of: infestation severity of each of said pet or fungus, humidity level and temperature level.

5. The method of claim 3, wherein said adjusting being by a non-linear optimization function.

6. The method of claim 3, wherein said non-linear optimization function being sigmoid based module.

7. A computer readable storage medium having encoded thereon computer executable instructions to cause one or more processing units to receive, from a user interface, one or more types of pests or fungus, ratio of one or more non-active ingredient and one or more parameters associated with environmental conditions or with said pest or fungus; for each of said one or more types of pests or fungus, retrieve from a data repository, one or more effective oil-types; retrieve from said data repository effective base ratio associated with each of said pest or fungus; adjust said effective base ratio in accordance with said one or more environmental parameters to thereby providing a plurality of adjusted effective base ratio; normalizing said plurality of adjusted effective base ratio; generate a data structure or software code; said data structure comprises said type of pest or fungus, said oil types associated with said normalized value and said one or more non-active ingredient in association with its ratio; said software code comprises instructions for generating labels for said active natural oil-based or instructions for generating said active natural oil-based; and transmit said data structure to a machine for generating labels for the product or for said machine for generating said active natural oil-based or transmitting said software code to a machine for generating labels for the product or said machine for generating said active natural oil-based.

8. A personalized composition comprising a plurality of oils, said oils being effective for a certain pest or fungus wherein ratio of each of said oils in said mixture being defined by effectiveness of said oil in treating said pest or fungus and further being defined by one or more parameters associated with environmental conditions or with said pest or fungus.

9. The composition of claim 8, wherein said one or more parameters comprises one member selected from a group consisting of: infestation severity of each of said pet or fungus, humidity level and temperature level.

10. A method comprising applying to a crop said composition of claim 8.
